(19) United States
(12) Patent Application Publication
(10) Pub. No.: US 2025/0029730 A1
(43) Pub. Date: Jan. 23, 2025

(54) SYSTEM AND METHOD FOR HEALTHCARE RISK ADJUSTMENT APPLICATIONS WORKFLOW TO ENHANCE ACCURACY AND EFFICIENCY WITH SCALABLE AND STACKED ARCHITECTURE

(71) Applicant: RAAPID, INC, Louisville, KY (US)

(72) Inventors: Durai Vaithilingam RAMACHANDIRAN, Thanjavur (IN); Raxitkumar GOSWAMI, Tragad (IN); Pinal PATEL, Mahesana (IN)

(73) Assignee: RAAPID, INC, Louisville, KY (US)

(21) Appl. No.: 18/780,157

(22) Filed: Jul. 22, 2024

Related U.S. Application Data
(60) Provisional application No. 63/527,833, filed on Jul. 20, 2023.

(51) Int. Cl. G16H 50/30; G16H 10/60; G16H 15/00

(52) U.S. Cl. CPC G16H 50/30; G16H 10/60; G16H 15/00

(57) ABSTRACT

Systems and methods for a risk adjustment solutions workflow for projecting a risk adjustment factor (RAF) are disclosed. The workflow comprises retrospective and prospective workflows which are stackable and modular in nature functioning as a standalone workflow. The workflow is provided with structured or unstructured data in the form of patient medical charts and the same is processed through an NLP module. The NLP module receives inputs from a knowledge graph to add a clinical context and relations with other entities found. An Application recommendation module suggests the output with a plurality of ICD-10-CM codes, description, diagnosis evidence, MEAT evidence, Cross-walk to HCC codes, and RAF score of HCC codes. The ICD-10-CM codes are compared with the claimed codes to determine a condition of proper claiming, under-claiming or over-claiming.

### FIELD OF INVENTION

The present invention relates to a clinical predictive and monitoring system for healthcare risk adjustment. More specifically, the present invention relates to a risk adjustment solution workflow for proactively identifying existing chronic conditions of a patient, potential care gaps, and suspected or overlooked emerging health conditions thereof for accurately projecting a risk adjustment factor.

### BACKGROUND OF THE INVENTION

The practice of medicine is characterized by increasingly overwhelming amount of information, new knowledge in diagnostics and therapeutics, and highly fragmented care environments, with potentially hundreds of different individuals delivering care to a single patient across healthcare settings.

The Medicare Advantage also known as Medicare Part C, is a health plan offered by private insurers in contract with the US federal government that enables Medicare-qualified people to take health insurance benefits. The Medicare Advantage (MA) program offers beneficiaries managed care alternatives, providing them with the option to choose private healthcare plans that cater to their evolving needs and enhance their healthcare experience. Enrollees in the MA program enjoy a range of benefits through contractual arrangements between the Centers for Medicare & Medicaid Services (CMS) and MA organizations, which act as intermediaries connecting beneficiaries with esteemed providers.

The federal requirements under the MA program stipulate that payments to MA organizations must be based on the anticipated cost of providing Medicare benefits to individual enrollees, while also accounting for variations in their demographic characteristics and health status. The CMS employs a two-component approach for calculating risk-adjusted payments. The base rate is determined through bids submitted by MA organizations, comparing them to specific benchmarks to establish the payment amount for each enrollee. Additionally, CMS calculates an individualized risk score for each enrollee based on their health status and demographic characteristics, reflecting the expected additional or reduced costs compared to the average. This risk adjustment methodology ensures fair and accurate payments, promoting appropriate reimbursement aligned with the actual costs incurred by MA organizations within the Medicare Advantage program.

Coordinating clinical decisions to advance the care and treatment of patients within this environment poses significant challenges. Hospital readmission is an important example of the type of problems health systems face in allocating critical resources in the midst of this fragmented environment.

If high-risk patients could be more easily and accurately identified at an early stage, the right interventions could be performed on the patients for which it is most needed, thereby lowering overall heart failure readmission rates. Today, most hospitals attempting to identify patients at risk are doing so manually, without leveraging the information available in their electronic health records. Hospital readmission is one of potentially thousands of adverse clinical events that could be prevented by electronic identification, targeting, coordinating and monitoring throughout the inpatient and outpatient environment. Thus, there is a need to identify risk stratify patients at the highest risk for hospital readmissions and other adverse clinical events.

To calculate an enrollee's risk score, the CMS utilizes diagnosis codes obtained from acceptable data sources, such as physicians and hospitals. These codes are mapped into Hierarchical Condition Categories (HCCs) based on clinical characteristics, severity, and cost implications. CMS consolidates certain HCCs into related-disease groups and assigns an HCC for the most severe manifestation within each group. Disease interactions, representing specific combinations of HCCs, receive separate factors to increase the enrollee's risk score. The risk adjustment program is prospective, with CMS using diagnosis codes from the service year to calculate risk scores for the following payment year. The risk score is additive, increasing as HCC factors accumulate, leading to higher monthly risk-adjusted payments to MA organizations. However, miscoded diagnoses or omissions by MA organizations can result in incorrect risk scores and improper payments from CMS.

The HCC coding plays a crucial role in the risk-adjustment payment model of the CMS, aimed at projecting future care costs for patients with chronic conditions. Accurate HCC coding provides a comprehensive understanding of a patient's health and ensures effective management through care programs. Improper coding can result in missed interventions and inadequate reimbursement. The HCC codes, when properly applied, can enhance health outcomes, reduce costs for health plans, and ensure appropriate compensation for high-risk patients. However, studies have shown inaccuracies in coding, with a significant percentage of claims having coding errors or missed opportunities. Health plans face challenges in meeting compliance requirements and dealing with increased audit volumes. Implementing best practices in HCC coding can help optimize reimbursement, reduce costs, and improve patient outcomes. Government audits, such as RADV and OIG audits, focus on validating data submitted by health plans for risk adjustment members, highlighting the importance of accurate coding and documentation.

The current reimbursement model poses challenges as monthly advance payments to organizations are based solely on projected costs, lacking adjustments to reflect the actual costs incurred. This results in potential financial uncertainties for MA organizations, with profits if actual costs are lower than payments or losses if costs exceed payments. Currently, there are no such solutions available to accurately project the risk adjustment costs indicating the reimbursement costs incurred by MA organizations so as to mitigate the financial risks.

To address the described issues, there is a need for novel approach to accurately project the risk adjustment costs by everaging advanced technologies and streamlining processes, the present invention aims to enhance accuracy, mitigate financial risks, optimize beneficiary care, and ensure appropriate reimbursement aligned with the actual costs incurred by MA organizations.

### SUMMARY OF THE INVENTION

In accordance with the present invention, systems and methods for a risk adjustment workflow to enhance accuracy and efficiency in projecting a risk adjustment factor are disclosed. The workflow comprises of an input stage, a processing stage and an output stage. The input stage is configured to receive one or more types of patient data. The processing stage is configured to review the patient data by comparing it with one or more claims reflecting the chronic health condition of the patient to identify a current chronic health condition of the patient as an output which is either properly claimed or under-claimed or over-claimed.

The workflow comprises retrospective and prospective workflows which are stackable and modular in nature functioning as a standalone workflow. The workflow is provided with structured or unstructured data in the form of patient medical charts and the same is processed through an NLP module. The NLP module receives inputs from a knowledge graph to add a clinical context and relations with other entities found. An Application recommendation module suggests the output with a plurality of ICD-10-CM codes, description, diagnosis evidence, MEAT evidence, Cross-walk to HCC codes, and RAF score of HCC codes. The ICD-10-CM codes are compared with the claimed codes to determine a condition of proper claiming, under-claiming or over-claiming.

### OBJECTIVES OF THE INVENTION

The main objective of the present invention is to provide a stackable system and method for healthcare risk adjustment applications workflow solution to enhance accuracy and efficiency in projecting a risk adjustment factor wherein each sub-solution is capable of being presented in a standalone manner. With this, the components, logic and flow may be reused and required specific features may be added as needed for the corresponding block/stack.

Another objective of the present invention is to provide a chart review workflow comprising processes of clinical input documents including unstructured and structured data to identify one or more clinical entities and their attributes, status, and context and produce the explainable output as suggestions to the user to curate them to make the final output, which provides both Potential Adds (under-claimed codes) and Potential Deletes (over-claimed codes) in the same tool.

Yet another objective of the present invention is to provide a prospective Pre-visit workflow by following the Chart Audit workflows with different sets of documents to identify the corresponding output.

Yet another objective of the present invention is to provide a (software as a service) SaaS system and APIs which have a built-in Compliance Guideline checking module and shows the warning message if it found any guideline violation, such as first name & last name mismatch between multiple documents, or date of birth missing in some documents, etc.

Yet another objective of the present invention is to provide a feedback collection and consolidation in the workflows is a novel method. It not only collects implicit and explicit feedback, but also consolidates the related feedback automatically, and then decides to act upon it. For example, by creating a new rule, and or injecting a new knowledge input to the system, etc.

Yet another objective of the present invention is to provide an important distinction is that the present AI is ‘explainable’; therefore auditable and easily validated. The suggested outputs of the present system of AI are not a black box AI output, but the user may intuitively understand the reasons behind the suggestion, with the evidence, linking the evidence in the medical charts & claims.

These and other objectives and advantages of the embodiments disclosed herein will become readily apparent from the following detailed description taken in conjunction with the accompanying drawings

### BRIEF DESCRIPTION OF DRAWINGS

The drawings constitute a part of this invention and include exemplary embodiments of the present invention illustrating various objects and features thereof.

FIG. 1: A simplified block diagram of a technical workflow and exemplary embodiment of a clinical predictive and monitoring system according to the present disclosure. These workflows may be stacked.

FIG. 2: Illustrates the Risk Adjustment Solutions workflow. It takes the unstructured document and structured data as input. They get passed into the NLP module and (International Classification of Diseases, Tenth revision, Clinical Modification) ICD-10-CM explainable system module. It also shows how the Knowledge Graphs are being used in this flow. Then, it sends the Optimal Codes as the Suggested (Identified) output for the end-users (human coders) interaction.

FIG. 3: Illustrates the workflow for the input of the claim data for comparison against the chart review output and categorize conditions as substantiated, unsubstantiated or unreported.

FIG. 4: Block illustrations of pre-visit solutions including a prospective Pre-visit solution and a Patient Stratification.

FIG. 5: Illustration of the high level workflow of the present invention.

FIG. 6: Illustration of the detailed workflow of the prospective Pre-visit solution and the Patient Stratification.

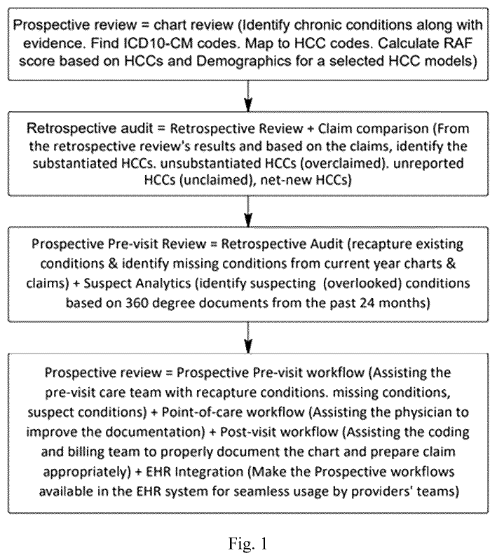

Although the specific features of the embodiments are shown in some drawings and not in others. This is done for convenience only as each feature may be combined with any or all of the other features in accordance with the embodiments herein.

### DETAILED DESCRIPTION OF THE INVENTION

The following description describes various features and functions of the disclosed system with reference to the accompanying figures. In the figures, similar symbols identify similar components, unless context dictates otherwise. The illustrative aspects described herein are not meant to be limiting. It may be readily understood that certain aspects of the disclosed system can be arranged and combined in a wide variety of different configurations, all of which have not been contemplated herein.

Accordingly, those of ordinary skill in the art will recognize that various changes and modifications of the embodiments described herein can be made without departing from the scope of the invention. In addition, descriptions of well-known functions and constructions are omitted for clarity and conciseness.

Features that are described and/or illustrated with respect to one embodiment may be used in the same way or in a similar way in one or more other embodiments and/or in combination with or instead of the features of the other embodiments.

The terms and words used in the following description are not limited to the bibliographical meanings, but, are merely used to enable a clear and consistent understanding of the invention. Accordingly, it should be apparent to those skilled in the art that the following description of exemplary embodiments of the present invention are provided for illustrative purposes only and not to limit the invention.

It is to be understood that the singular forms “a”, “an” and “the” include plural referents unless the context clearly dictates otherwise.

It should be emphasized that the term “comprises/comprising” when used in this specification is taken to specify the presence of stated features, integers, steps or components but does not preclude the presence or addition of one or more other features, steps, components or groups thereof.

The present invention introduces a stackable modular workflow for risk adjustment, including chart review, chart audit, and pre-visit audit workflows to enhance accuracy, mitigate financial risks, optimize beneficiary care, and ensure appropriate reimbursement aligned with the actual costs incurred by Medicare Advantage (MA) organizations.

The risk adjustment solutions provided by the present invention, encompasses two major workflows including a first workflow and a second workflow, each with its own subdivisions. In some embodiments, the first workflow is a retrospective workflow and the second workflow is a prospective review workflow. By offering comprehensive solutions within the retrospective and prospective review workflows, the risk adjustment process can be optimized to facilitate accurate data submission, enhance patient care, and support the efficient operations of both the health plans and the healthcare providers. A comprehensive overview of various risk adjustment workflows and their solutions is mentioned below in Table 1.

TABLE 1

| Solution | Sub-solution | Definition |
|---|---|---|
| Retrospective | Chart Review | Analyze one year medical charts to |
| Workflow | | identify the ICD-10-CM codes |
| | | (thereby HCC codes) along with |
| | | supporting evidence from charts per |
| | | each MA patient. |
| | Chart Audit | Analyze one year medical charts to |
| | | identify the ICD-10-CM codes |
| | | (thereby HCC codes) along with |
| | | supporting evidence from charts per |
| | | each MA patient; |
| | | Further, compare it with the |
| | | corresponding one year provider |
| | | claims to identify whether the codes |
| | | identified are properly claimed, |
| | | underclaimed or overclaimed. |
| | | a) Properly claimed codes: |
| | | Substantiated codes = Diagnosis and |
| | | evidence found in medical charts and |
| | | also found in provider claims. |
| | | b) Underclaimed codes: Missed codes = |
| | | Diagnosis and evidence found in |
| | | medical charts but not found in |
| | | provider claims. These codes are |
| | | considered for “potential add” during |
| | | Retrospective Review. |
| | | c) Overclaimed codes: |
| | | Unsubstantiated codes = Diagnosis |
| | | and evidence not found in medical |
| | | charts, but found in provider claims. |
| | | These codes are considered for |
| | | “potential delete” during |
| | | Retrospective Review. |
| Prospective | Pre-visit | Analyze current year (up-to-date) |
| Review | Review | medical charts and claims to identify |
| | | the ICD-10-CM codes (thereby HCC |
| | | codes) along with supporting evidence |
| | | from charts and claims per each MA |
| | | patient, and three categories of codes |
| | | are generated. |
| | | a) The codes are categorized as |
| | | “Recapturing existing conditions” if |
| | | the evidence found in both medical |
| | | charts and claims. |
| | | (This is equivalent to properly claimed |
| | | codes in Chart Audit solution, except |
| | | the DoS range is current year instead |
| | | of past years). |
| | | b) If the evidence is found only in |
| | | medical charts, but not in provider |
| | | claims, then those codes are |
| | | categorized as “Recapturing |
| | | opportunity conditions”. |
| | | (This is equivalent to under-claimed |
| | | codes in the Chart Audit solution, |
| | | except the DoS range is current year |
| | | instead of past years). |
| | | c) If the evidence is found only in the |
| | | provider claims but not in the medical |
| | | charts, then those codes are |
| | | categorized as “Recapturing risk |
| | | conditions”. |
| | | (This is equivalent to over-claimed |
| | | codes in the Chart Audit solution, |
| | | except the DoS range is current year |
| | | instead of past years). |
| | | Analyze the last 2 years medical |
| | | charts, provider claims, prescription |
| | | claims, lab reports, and any other |
| | | medical documents and find out the |
| | | chronic conditions that are found from |
| | | the medical charts & claims (so-called |
| | | properly claimed codes), and chronic |
| | | conditions found only in the medical |
| | | charts but not found in claims (so- |
| | | called underclaimed codes) and |
| | | deduct the codes that are found in the |
| | | current year medical charts/claims |
| | | (recapturing existing conditions, |
| | | recapturing opportunity conditions) |
| | | are categorized as “Suspecting Care |
| | | Gaps” conditions. |
| | | Also, by analyzing the last 2 years and |
| | | current year all medical data (360 |
| | | degree longitudinal data), using |
| | | clinical Knowledge Graph technology, |
| | | the system will be able to find any |
| | | abnormal indicators and connect them |
| | | together to suggest “Suspecting |
| | | Overlooked Conditions”. These |
| | | chronic conditions are not directly |
| | | caught and treated by any physicians |
| | | for this patient at least in the last 2 |
| | | years and the current year until now. |
| | | Such conditions need to be validated |
| | | by the physicians in the upcoming |
| | | patient encounter (i.e., face-to-face |
| | | visit). |
| | Point-of-care | Solution to augment physician and |
| | Review | clinical staff to increase their |
| | | documentation completeness, |
| | | increasing their accuracy in |
| | | documenting, and adding more |
| | | specific details in their documentation |
| | | during the encounter. |
| | Post-Visit | Solution to augment physician and |
| | Review | clinical staff to increase their |
| | | documentation completeness, |
| | | increasing their accuracy in |
| | | documenting, adding more specific |
| | | details in their documentation post |
| | | encounter, and also do the concurrent |
| | | coding for the chart, so that it could |
| | | go to the billing/claiming team to make |
| | | the optimized claims. |

In some embodiments, the retrospective workflow is provided for payers such as Medicare and health plan providers to accurately identify and submit one or more chronic health conditions of a patient corresponding to one or more Hierarchical Condition Category (HCC) codes as a part of the Risk Adjustment Data submission for Medicare Advantage (MA) members. It may be appreciated that HCC codes are medical codes describing a group of similar diagnoses of chronic or severe health conditions of a patient for projecting a risk adjustment cost factor. HCC codes are used by MA members to estimate health costs for a patient in a current year. Compliance with this requirement is mandatory for health plans managing MA members.

In some embodiments, the retrospective workflow further comprises chart review and chart audit solutions, each configured to facilitate a thorough analysis of a patient's records.

Chart review: The Chart review refers to the analysis of at least one year of medical charts of a patient to identify morbidity data including one or more ICD-10-CM codes (International Classification of Diseases, Tenth revision, Clinical Modification), and thereby the corresponding one or more HCC codes along with supporting evidence from the medical charts according to each MA patient are identified. The morbidity data includes one or more ICD codes to identify a patient's diseases and medical conditions and said codes are maintained by the World Health Organization. ICD-10-CM denotes the revised (tenth) version of such codes.

Chart audit: The Chart audit refers to the analysis of at least one year of medical charts to identify one or more ICD-10-CM codes and thereby the corresponding one or more HCC codes along with supporting evidence from the medical charts for each MA patient. Further, the identified HCC codes are compared with the corresponding one year of provider claims i.e., the claims provided by a healthcare provider in order to identify whether the said identified codes are properly claimed, under-claimed or over-claimed:
a. Properly claimed codes: The properly claimed codes are the codes which are substantiated by the said analysis, i.e., evidence and diagnosis are found in the analyzed medical charts and as well as in the provider claims.
b. Under-claimed codes: The under-claimed codes refer to the codes which have been missed, i.e., evidence for the code has been found in the medical charts but the same code is missing in the provider claims. The said under-claimed codes are considered for “potential add” for the retrospective review stage.
c. Over-claimed codes: The over-claimed codes refer to the codes which are unsubstantiated by the medical charts i.e., the said codes were not diagnosed but were found in the provider claims. The said over-claimed codes are considered for “potential delete” during the retrospective review stage.

In some embodiments, the prospective review workflow is provided for payers and health plans to proactively identify or evaluate a summary of a patient's existing chronic conditions, potential care gaps, and suspected or overlooked conditions. The prospective review workflow may be subdivided into a pre-visit, a point-of care, and a post-visit solution. This evaluation is conducted prior to the patient's face-to-face encounter with a physician, enabling the physician to deliver effective and value-based care. The prospective review workflow may also include one or more solutions including a point-of-care solution to provide assistance during the patient encounter and a post-visit solution to ensure accurate and error-free documentation, thereby enabling providers to perform their clinical responsibilities efficiently and productively.

The prospective review workflow comprises the following activities:
Pre-visit review: The Pre-visit review refers to the analysis of the past two years and the current year up-to-date medical charts including but not limited to claims, lab reports, radiology reports, pharmacy reports or any other related medical documents, to identify one or more ICD-10-CM codes and thereby the corresponding one or more HCC codes along with the supporting evidence from said input documents for each MA patient. The said review may generate any of the three categories of the following codes:
a. Recapturing existing conditions-If the evidence for a code is found in both the medical charts and the claims then said code is categorized under the recapturing existing conditions. The recapturing existing condition is equivalent to the properly claimed codes in the Chart Audit workflow or solution, except a Date of Service (DOS) range is the current year instead of the past years.
b. Recapturing opportunity conditions-If the evidence for a code is only found in the medical charts but not in the provider claims then the said code is categorized under the recapturing opportunity conditions. The recapturing opportunity condition is equivalent to the under-claimed codes in the Chart Audit workflow, except the DoS range is the current year instead of the past years.
c. Recapturing risk conditions-If the evidence for a code is found only in the provider claims but not in the medical charts, then the said codes are categorised as the recapturing risk conditions. The recapturing risk condition is equivalent to the over-claimed codes in the Chart Audit workflow, except the DoS range is the current year instead of the past years.

The pre-visit review analyses the medical charts, provider claims, prescription claims, lab reports, and any other medical documents as a 360 degree longitudinal data from the last 2 years to find out the chronic conditions that are present in the said medical charts and claims, i.e. the properly claimed codes, the chronic conditions found only in the medical charts but not found in the claims, which are the under-claimed codes and deduct the said codes that are found in the current year's medical charts and claims which is the recapturing existing conditions and the recapturing opportunity conditions. The said codes are categorized as Suspecting Core Gaps (code gaps) conditions.

Based on said analysis of 360 degree longitudinal data of the past 2 years and the current year using a Clinical Knowledge Graph technology, the present invention locates any abnormal indicators and connects them together to suggest “Suspecting Overlooked emerging conditions”. These are the chronic conditions that are not directly caught and treated by any physicians for the patient at least in the last 2 years and the current year until now. Such conditions are required to be validated by the physicians in the upcoming face-to-face patient encounter. The Clinical Knowledge Graph technology implemented in the present invention is infused with a Natural Language Processing module to understand the clinical entities along with their context and meaning, and also it helps to navigate the connected tree through the entities from multiple directions. It may be appreciated that the Knowledge Graph technology is utilized for organizing information in a structured way by explicitly describing the relations among entities. It is a large network of entities (subjects, objects), and their properties (relationships) between entities. The entities connect with each other through various relationships. Its goal is to not just represent information, but always to make sense and meaning of its data.

The Point-of-care review is a solution to augment the physician and the clinical staff to increase the completeness of their documentation thus increasing their accuracy in documenting, adding more specific details in their documentation post encounter.

The Post-visit review solution augments the physician and clinical staff to improve their documentation completeness increasing their accuracy in documenting and adding more specific details in their documentation post-encounter. Further, concurrent coding and auditing for the chart are performed so that it may go to the billing or claiming team to determine optimized claims.

In another embodiment of the present invention, said workflows are stacked to provide for a comprehensive workflow comprising:
Retrospective Review or Chart review: Identifying chronic health conditions of a patient along with evidence, finding one or more ICD 10-CM codes, mapping said ICD-10-CM codes to the corresponding one or more HCC codes, and optionally calculating a Risk Adjustment Factor (RAF) score based on the HCC codes and demographics for a selected HCC model.
Retrospective audit or Chart audit i.e., retrospective review along with claim comparison: Identifying the substantiated HCC, unsubstantiated HCCs (over-claimed), unreported HCCs (unclaimed) and net-new HCCs from the retrospective review results and the claims based on the same.
Prospective pre-visit review, i.e., Retrospective audit: Recapturing of the existing conditions and identifying missing conditions from the current year charts and claims. Further, suspect analytics relates to identifying suspecting or overlooked conditions based on the 360-degree documents from the past 24 months and more.
Prospective review, i.e., prospective pre-visit workflow: Assisting the pre-visit care team with recapture conditions, missing conditions, suspect conditions, and point-of-care workflow which relates to assisting the physician to improve the documentation and post-visit workflow which involves assisting the coding and billing team to properly document the chart and prepare claim appropriately. Further, an Electronic Health Record (EHR) integration includes the making of prospective workflows available in the EHR system for seamless usage by providers' teams.

A schematic representation of the said stacked workflows may be described as follows:
Retrospective Review=A
Retrospective Audit (B)=Retrospective Review (A)+Claim Comparison (a)=A+a
Prospective Pre-visit (C)=Retrospective Audit (B)+Suspect Analytics (b)=B+b
Prospective Solution (D)=Prospective Pre-visit (C)+Point of care and Post-visit solution (c)=C+c=B+b+c=A+a+b+c.

In some embodiments, the workflow components of the present invention are described in detail below in accordance with FIGS. 2 to 6.

### Chart Review Workflow

In accordance with FIG. 2, the Risk Adjustment Solutions workflow is described. It takes the unstructured document and structured data as input. The data is passed into a Natural Language Processing (NLP) module and an Application Recommendation System module. The NLP module is configured to take unstructured data as input and understands the section, sentence, entities, metadata or attributes, status, and negation and then it normalizes depending on the use case. The Application Recommendation System is an application layer in the present workflow which puts multiple things together for the specific use case. In an embodiment, the Application Recommendation System is an ICD-10-CM system module. The Application Recommendation Module is configured to make the present AI-based system an explainable AI module, i.e., the AI system is not a black-box system in the workflow, rather it is explained with supporting evidence and justification on why a particular diagnosis (ICD-10-CM code) is suggested by this system. It also shows how the Knowledge Graphs are being used in this flow. The Application Recommendation System provides the Optimal Codes as a Suggested (Identified) output for further verification and approval by a coder, wherein the coder is a human coder.

The Coders (i.e., Human-in-the-loop) interact (accept, update, ignore, reject, add) with the suggested output and get a curated output. In response, a feedback is collected from such interactions which is provided to a Feedback Processing module which is configured to decide on how to handle the received feedback and utilize it accordingly for further training & fine-tuning of the disclosed Artificial Intelligence based Machine Learning or Deep Learning models.

In some embodiments, patient data sources may include non-healthcare entities. These are entities or organizations that are not thought of as traditional healthcare providers. These entities may provide non-clinical data that include, for example, gender; marital status; education; community and religious organizational involvement; proximity and number of family or caregiving assistants; address; census tract location and census reported socioeconomic data for the tract; housing status; number of housing address changes; frequency of housing address changes; requirements for governmental living assistance; ability to make and keep medical appointments; independence on activities of daily living; hours of seeking medical assistance; location of seeking medical services; sensory impairments; cognitive impairments; mobility impairments; educational level; employment; and economic status in absolute and relative terms to the local and national distributions of income; climate data; and health registries. Such data sources may provide further insightful information about patient lifestyle, such as the number of family members, relationship status, individuals who might help care for a patient, and health and lifestyle preferences that may influence health outcomes.

In a preferred embodiment, a method for the chart review workflow comprises the following steps:
1. Inputting of the documents in the form of a patient medical chart wherein the said input may include: unstructured documents such as PDF files (machine-readable, scanned files, faxed files) of medical charts; or structured data in HL7, FHIR, CCDA format, pulled from HER (Electronic Health Record) system, HIE (Health Information Exchange) system, from third party Health IT companies. The said input is passed into the NLP pipeline to convert unstructured information into structured format. In this step, if the input file is in PDF format, the said file is converted into a text format using an OCR (Optical Character Recognition) engine.
2. The said inputted documents are passed into the Application Recommendation system which is the ICD-10-CM code recommendation system to identify the evidence from the patient's unstructured report (input text) and make the system explainable.
3. Input is taken from the master knowledge graphs for the NLP module to add clinical context and relations with other entities found.
4. The output from the NLP module is given to the Application module.
5. Input is added from the ICD-10-CM knowledge graph to add context to the findings of the Application module.
6. The output of the said application module suggests the ICD-10-CM Codes, description, diagnosis evidence, (Monitor, Evaluate, Assess, and Treat) MEAT evidence, Cross-walk to HCC codes, and optionally RAF score of HCC codes.
7. The human-in-the-loop (Coder, reviewer, auditor) checks the suggested output and performs one of the following curation tasks or a combination thereof:
   a. Acceptance of the suggested output as it is;
   b. Updating of the suggested output, for example, editing of the code, addition of evidence or removal of evidence;
   c. Rejection of the suggested output; and
   d. Addition of new code and supporting evidence from the medical charts.
8. During the curation activities of the previous step, the feedback is collected and sent to the feedback processing module wherein the feedback comprises the following types of feedback:
   a. Implicit feedback-automatic collection of the feedback while the human performs accept, update, and add actions.
   b. Explicit feedback-explicit collection by asking for the feedback (reason) while the human performs the deletion or rejection action.
9. The feedback is processed by the feedback processing module and it is decided to update the application module according to the consolidated feedback with specific action including adding a new rule, fine-tuning a model, etc.

### Chart Audit Workflow

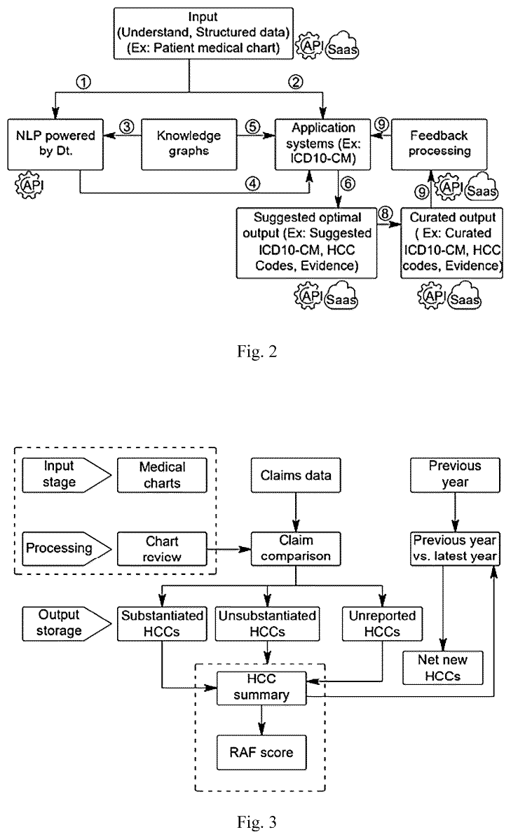

In accordance with FIG. 3, the Chart Audit workflow is provided along with the Chart Review workflow for processing the Claim Comparison steps.

Health plans can generate more accurate reimbursements by reviewing claim data from the previous year. When compared to outcomes of the chart review, this data helps plans to delete previously submitted diagnosis codes that were not substantiated. It also enables them to add appropriate codes found within the records.

The claims data may be in the form of one or a combination of the following:
a. Provider claims
   i. Form 837-I
   ii. Form 837-P
b. CMS MAO acknowledgement to Health plan
   i. MAO-004
c. First level coding results
   i. ICD-10-CM codes according to a chart in a spreadsheet or any other custom agreed format.
      The MAO-004 report is intended for Medicare Advantage Organizations (MAOs) and other entities participating in the Medicare Advantage program about the risk adjustment eligibility of diagnoses submitted on Encounter Data and Chart Review records if a given diagnosis code submitted is eligible for risk adjustment.

The Chart Audit workflow provides output codes in three categories as explained in Table 2 below:

TABLE 2

| | | | Suggested Output with |
|---|---|---|---|
| Medical | | | Code Category (after |
| Chart | Claim | Code Status | Claim Comparison) |
| Yes | Yes | Matched | Properly claimed code; |
| | | | Substantiated code |
| Yes | No | Missed | Under-claimed code; |
| | | (to claim) | Unclaimed code; |
| | | | Missed code; |
| | | | Code for potential Add |
| | | | in RA review |
| No | Yes | Extra | Over-claimed code; |
| | | | Unsubstantiated code; |
| | | | Code for potential Delete |
| | | | in RA review |

As tabulated above, if a particular code is present in the medical chart and as well as in the claim, then the said code is considered to be a properly claimed code or substantiated code. If a particular code is present in the medical chart but not present in the claim, then the said code is considered to be an under-claimed code or unclaimed code or missed code or a code for potential add in the RA review. This process and comparison will be done for all Date of Service and finally, the unique codes will be calculated member-wise.

### Pre-Visit Audit Workflow

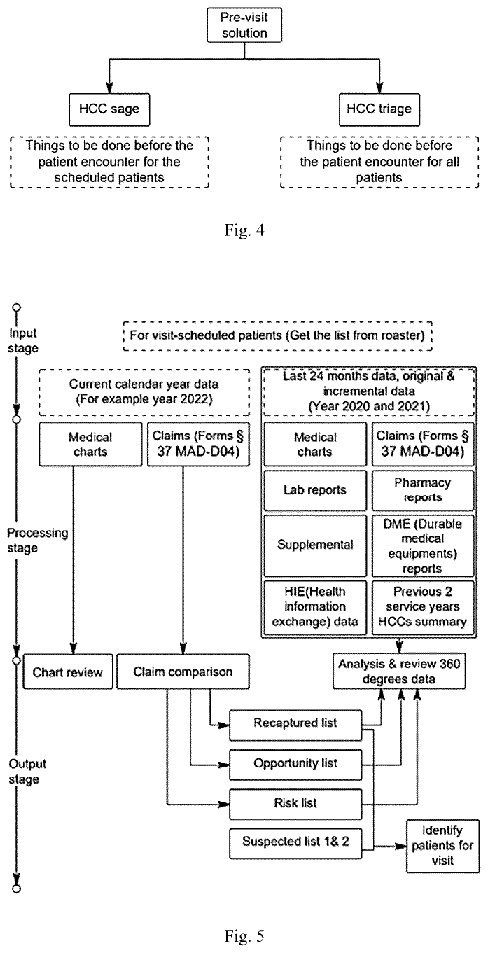

In accordance with FIG. 4, prior to a patient's visit to the provider, the Pre-visit workflow prepares a summary of the details of the said patient to supplement the efforts of the Pre-care team and the Provider.

The entire workflow as described in FIG. 4 can be implemented for the already scheduled patients or also for the entire patient list depending on the use case of a Provider or Payer.

The prospective pre-visit product runs the process for the already scheduled patients. It includes the things to be done before the patient encounter for the scheduled patients.

The patient stratification runs the same process for the entire patients and prioritizes the patients based on given guidelines. It includes the things to be done before the patient encounter for all patients.

As shown in FIG. 3, the clinical predictive and monitoring system may receive data streamed in real-time, or from historic or batched data from various data sources. Further, the system may store the received data in a data store or process the data without storing it first. The real-time and stored data may be in a wide variety of formats according to a variety of protocols, including but not limited to CCD, XDS, HL7, SSO, HTTPS, EDI, CSV, etc. The data may be encrypted or otherwise secured in a suitable manner. The data may be pulled (polled) by the system from the various data sources or the data may be pushed to the system by the data sources. Alternatively or in addition, the data may be received in batch processing according to a predetermined schedule or on-demand. The data store may include one or more local servers, memory, drives, and other suitable storage devices. Alternatively or in addition, the data may be stored in a data center in the cloud.

### Inputs and Outputs

The possible input and output for pre-visit solutions are described below in Table 3:

TABLE 3

| I/O | Pre-visit Solution |
|---|---|
| Input | 360-degree longitudinal data of the patient for the |
| | last 2 or more years and the current year till now. |
| | Medical Charts |
| | Provider Claims |
| | CMS Claim Acknowledgements |
| | Prescription Claims/Medication reports |
| | Lab reports |
| | Radiology reports |
| Output | Summary of the diagnosis Codes with below categories |
| | per patient for Pre-care term/Coders to verify: |
| | Recapturing Existing conditions |
| | Recapturing Under-claimed conditions |
| | Recapturing Over-claimed conditions |
| | Suspecting Care gaps (code gaps) conditions |
| | Suspecting Overlooked emerging conditions |

The various possible outputs as mentioned, are described as follows:
1. Recapturing Existing Conditions: Includes the conditions that are captured properly in the current year's medical charts and the corresponding claims. This is similar to the Retrospective Audit solution's Properly Claimed Conditions but based on the current year's data, specific to DoS.
2. Recapturing Under-claimed Conditions (Opportunity): Includes the conditions that are captured properly in the current year's medical charts but not found in the corresponding claims. This is similar to the Retrospective Audit solution's Under-claimed Conditions except based on the current year's data, specific to DoS.
3. Recapturing Over-claimed Conditions (Risk): The conditions that are not captured in the current year's medical charts but are found in the corresponding claims. This is similar to the Retrospective Audit solution's Over-claimed Conditions but based on the current year's data, specific to DoS.
4. Suspecting Care Gaps Conditions: The conditions that are captured properly in the previous two years' medical charts and the corresponding claims or maybe under-claimed; But not captured in the above-mentioned first and second categories. This is similar to the Retrospective Audit solution's Properly Claimed Conditions and Under-claimed Conditions but based on the last 2 years data and without the conditions of the lists under the first and the second categories, specific to DoS.
5. Suspecting Overlooked Conditions: The conditions that are not captured directly in the medical charts for the past 2 years along with the current year. These are the truly overlooked conditions. Suspecting these conditions by connecting the indicators or abnormalities found across the data, i.e., connecting the dots, not specific to DoS.

An example of an output is described below in Table 4:

TABLE 4

| | | Last 2 | Last 2 | |
|---|---|---|---|---|
| | Current | Years | Years | |
| Current Year | Year | Medical | Claims, | Diagnosis |
| Medical Charts | Claims | Charts | Others | Categories |
| CKD | CKD | — | — | Recapturing |
| | | | | Existing |
| | | | | Conditions: |
| | | | | CKD |
| Hypertension | — | — | — | Recapturing |
| | | | | Under-claimed |
| | | | | Conditions: |
| | | | | Hypertension |
| — | CHF | — | — | Recapturing |
| | | | | Over-claimed |
| | | | | Conditions: |
| | | | | CHF |
| — | — | CKD, | CKD, | Suspecting |
| | | COPD, | Diabetes, | Care Gaps |
| | | Diabetes, | Hypertension | Conditions: |
| | | Hypertension | | Diabetes, |
| | | | | COPD |

| | |
|---|---|
| Obstructive Sleep Apnea | Suspecting |
| | Overlooked |
| | Conditions: |
| | Obstructive |
| | Sleep Apnea |

### High Level Workflow

The higher level workflow, as illustrated in FIG. 5, comprises three major stages of an Input stage, a Processing stage, and an Output stage.

### Input Stage:

The input stage broadly takes the current year medical data along with the last 2 years' medical data.

The current year medical charts and claims are used to locate the chronic conditions that are existing (and captured) in the current year medical reports.

The last 2 years medical charts, claims, and other documents are used to identify the suspecting care gaps conditions and suspecting overlooking conditions.

### Processing Stage:

As described in the Stacked Development approach, this uses multiple Retrospective Chart Audit workflows to achieve the final results.
The first Audit workflow is to run with the current year's medical charts and claims.
   Here the results will be recapturing existing conditions, recapturing opportunity conditions, and recapturing risk conditions.
   For the very first time (say the very first visit of the year), obviously there won't be any documents to review from the current year. So, this list will be empty.
The second workflow is to run the Audit workflow with the last 2 years medical charts and claims.
   Now, this will also give Properly claimed conditions, Unclaimed conditions and Over-claimed conditions.
   Optionally it may ignore the Over-claimed conditions.
   Take the Properly claimed and Under-claimed conditions.
   Now deduct the conditions that come from the first audit workflow. The remaining conditions are Suspect Care Gap conditions.
The third workflow relates to running an entire data set to identify suspecting overlooking conditions, which means to suspect chronic conditions based on indicators identified through the Knowledge Graph.

### Output Stage:

By following the above processing, it gives the outputs into the corresponding categories as explained in the Output Description section.

Patient stratification is provided to prioritize the patients, after the above processing, it can do the sorting based on the below guidelines.

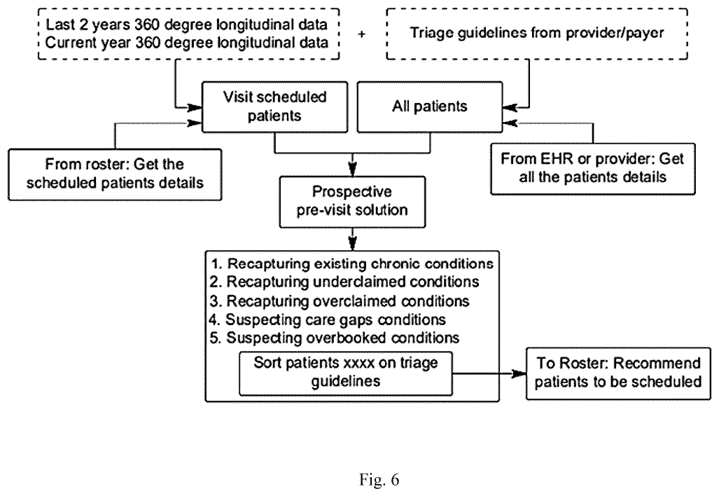

The guidelines may be:
a) More sick patients (based on the total number of HCC conditions) as illustrated in FIG. 6.
b) More RAF score patients (if the system gets the Plan details to calculate the RAF score)
c) More Recaptured Opportunity conditions+Suspecting Care Gaps (code gaps)+Suspecting Overlooked (emerging) conditions
d) More Suspecting Care Gaps (to address them quickly), etc.

An embodiment of the present invention comprises a data store operable for receiving and storing data associated with patients selected from medical and health data; and several social, behavioral, lifestyle, and economic data; at least one health plan to accurately identify and submit chronic conditions (HCC codes) as part of their Risk Adjustment Data submission for Medicare Advantage (MA) members; at least one workflow for chart review and chart audit solutions, facilitating thorough analysis of patient records and identify at least one high-risk associated with the patient, at least one medical condition; a prospective review for providers, payers, and health plans to proactively identify a summary of a patient's existing chronic conditions, potential care gaps, and suspected or overlooked conditions; information to intervention coordination in terms of pre-visit, point-of-care, and post-visit solutions about the identified risks.

The stacked approach of the present invention is a novel and modular method to build a comprehensive solution while each sub-solution may be presented and commercialized separately. With this, the components, logic and flow may be reused and more specific features may be added as required for the corresponding block or stack. The entire chart review workflow is a novel workflow which processes any type of clinical input documents (unstructured and structured) to identify the clinical entities, and their attributes, status, context and produce the explainable output as suggestions to the user to curate them to make the final output. The present system provides both Potential Adds (under-claimed codes) and Potential Deletes (over-claimed codes) in the same tool. In an embodiment, the retrospective workflow and the prospective workflow are connected as modular stack block layers, wherein said each workflow is configured to operate separately as an individual stack block. The retrospective and prospective workflow solutions are built using a stackable architecture model with reusable components and reusable modules and systems.

Prospective Pre-visit workflow by following the Chart Audit workflows with different sets of documents to identify the corresponding output presents another novel feature of the present invention. The present Saas (software as a service) system and the respective APIs have a built-in Compliance Guideline checking module and show a warning message if it finds any guideline violation, such as first name & last name mismatch between multiple documents, or date of birth missing in some documents, etc.

The feedback collection and consolidation in the workflows relates to not only collecting implicit and explicit feedback, but also consolidating the related feedback automatically, and then deciding to act upon it. For example, by creating a new rule, and or injecting a new knowledge input into the system, etc. An important distinction is that the present AI is ‘explainable’; therefore auditable and easily validated. The suggested outputs of the present invention are not a black box AI output, but the user is enabled to intuitively understand the reasons behind the suggestion, with the evidence, linking the evidence in the medical charts & claims.

In some embodiments, a computer-implemented method for calculating a risk adjustment factor (RAF) by a risk adjustment workflow system is disclosed, comprising: receiving an input including a structured data and an unstructured data by a retrospective workflow to identify one or more clinical entities; parsing the unstructured data format into the structured data format by a Natural Language Processing (NLP) module and passing said parsed structured data and the input unstructured data to an Application Recommendation System; identifying, based upon the unstructured data, an evidence by the Application Recommendation System and processing said evidence based on an input received from a master knowledge graph module; providing, based on the processing by the Application Recommendation System, an output by a prospective workflow to a coder, wherein the coder performs a plurality of curation tasks to provide a feedback to a feedback processing module; and updating, based on the feedback received by the coder, the Application Recommendation System by adding a new rule therein and fine tuning the risk adjustment workflow system.

The retrospective workflow further comprises: analysing a previous health data of the preceding two years to identify one or more previous Hierarchical Category Codes (HCC) codes from one or more morbidity (ICD-10-CM) codes by a chart review component; and identifying, based on a comparison of the identified previous HCC codes with one or more previously filed claims, a claim condition selected from a properly claimed condition, an unclaimed condition, or an overlooked claimed condition, by a chart audit component. The prospective workflow comprises: analysing a current health data of the current year to identify one or more current HCC codes from one or more ICD-10-CM codes and one or more previously filed claims; and identifying, based on a comparison of the identified current HCC codes with one or more previously filed claims, a chronic health condition selected from an existing condition, an opportunity condition, and a risk condition. The retrospective workflow and the prospective workflow are connected as modular stack block layers, wherein said each workflow is configured to operate separately as an individual stack block.

In some embodiments, a system for calculating a risk adjustment factor (RAF), comprising one or more processors and one or more computer-readable storage devices having stored a plurality of computer-executable instructions is disclosed, wherein execution of the computer-executable instructions causing the computer system to: receive an input including a structured data and an unstructured data by a retrospective workflow to identify one or more clinical entities; parse the unstructured data format into the structured data format by a Natural Language Processing (NLP) module and said parsed structured data and the input unstructured data is passed to an Application Recommendation System; identify an evidence by the Application Recommendation System based upon the unstructured data and said evidence is processed based on an input received from a master knowledge graph module; suggest an output by a prospective workflow to a coder, wherein the coder performs a plurality of curation tasks to provide a feedback to a feedback processing module; and update the Application Recommendation System by adding a new rule therein and fine tuning the risk adjustment workflow system, wherein the retrospective workflow and the prospective workflow are provided as at least two stack block layers connected with each other and each stack block comprises a plurality of components, logic and flow.

In some embodiments, provided herein are systems and methods for healthcare risk adjustment workflow solutions for calculating a risk adjustment factor (RAF) that may be implemented using hardware, software, firmware, and/or any combination thereof. The workflow solutions may include one or more sub-workflow solutions configured to execute one or more programs to generate the risk adjustment factor. The workflows and processes may be performed by general or special-purpose computing systems. In an exemplary configuration, the embodiments disclosed herein may be implemented using a container orchestration system such as Kubernetes deployed on a virtual machine. This exemplary configuration may include a B-Series burstable Virtual Machine, e.g., Azure Standard_B4 ms (4vCPU, 16 GB Memory) wherein the majority of services are implemented using Azure Kubernetes Service. In said deployment environment, following is the configuration: Load balancing for distributing network traffic: Standard/WAF V2; Cognitive Search: Standard/OCR S1; Server 1: General Purpose, D2ds_v4, 2 vCores, 8 GiB RAM, 64 storage, 1000 IOPS, Server 2: General Purpose, D8ds_v4, 8 vCores, 32 GiB RAM, 64 storage, 1000 IOPS; Storage: Storage V2, Relational Database; Message broker: Standard/Premium; Key Management: Standard; and Log Management: Standard. It may be appreciated that the embodiments disclosed herein may also be implemented on some other alternative configurations which may vary according to the load and the specific deployment requirements.

While this invention has been described in connection with what is presently considered to be the most practical and preferred embodiment, it is to be understood that the invention is not limited to the disclosed embodiments, but, on the contrary, is intended to cover various modifications and equivalent arrangements included within the scope of the appended claims.

What is claimed is:

1. A computer-implemented method for calculating a risk adjustment factor (RAF) by a risk adjustment workflow system, comprising:
receiving an input including a structured data and an unstructured data by a retrospective workflow to identify one or more clinical entities;
parsing the unstructured data format into the structured data format by a Natural Language Processing (NLP) module and passing said parsed structured data and the input unstructured data to an Application Recommendation System;
identifying, based upon the unstructured data, an evidence by the Application Recommendation System and processing said evidence based on an input received from a master knowledge graph module;
providing, based on the processing by the Application Recommendation System, an output by a prospective workflow to a coder, wherein the coder performs a plurality of curation tasks to provide a feedback to a feedback processing module; and
updating, based on the feedback received by the coder, the Application Recommendation System by adding a new rule therein and fine-tuning the risk adjustment workflow system.

2. The method of claim 1, wherein the retrospective workflow comprises the steps of: analysing a previous health data of the preceding two years to identify one or more previous Hierarchical Category Codes (HCC) codes from one or more morbidity (ICD-10-CM) codes by a chart review component; and
identifying, based on a comparison of the identified previous HCC codes with one or more previously filed claims, a claim condition selected from a properly claimed condition, an unclaimed condition, or an overlooked claimed condition, by a chart audit component, and
the prospective workflow comprises the steps of: analysing a current health data of the current year to identify one or more current HCC codes from one or more ICD-10-CM codes and one or more previously filed claims; and
identifying, based on a comparison of the identified current HCC codes with one or more previously filed claims, a chronic health condition selected from an existing condition, an opportunity condition, and a risk condition.

3. The method of claim 2, wherein the retrospective workflow and the prospective workflow are connected as modular stack block layers, wherein said each workflow is configured to operate separately as an individual stack block.

4. The method of claim 2, wherein the previous and current health data is the health data of a patient including one or more documents selected from a list of documents including medical charts, lab reports, radiology reports, claims including MAO-004 forms, pharmacy reports, durable medical equipment (DME) reports, health information exchange (HIE) data, and Hierarchical Confirmation Code (HCC) summary.

5. The method of claim 1, wherein the input data of the master knowledge graph is processed by the NLP module to add clinical context and relations with a plurality of entities provided by the master knowledge graph.

6. The method of claim 1, wherein the unstructured and structured data include clinical and non-clinical data of a patient, the clinical data include health records and the non-clinical data include one or more demographic details of the patient, wherein the unstructured data includes machine-readable and scanned electronic documents, and the structured data comprises health record data in HL7, FHIR, CCDA format retrieved from Electronic Health Record (HER) system and Health Information Exchange (HIE) system.

7. The method of claim 1, wherein the Application Recommendation System is an ICD-10-CM code recommendation system.

8. The method of claim 1, wherein the coder performs one or more curation tasks selected from the tasks of accepting the output provided by the prospective workflow; updating the output including editing of the code, addition of evidence or removal of evidence; rejecting the suggested output; adding new code and supporting evidence from the medical charts.

9. The method of claim 1, wherein, based on one or more curation tasks executed by the coder, the feedback processing module provides one or more feedbacks, including:
an implicit feedback, based on the curation tasks including accepting, updating, and adding of actions, automatically collects the feedback; and
an explicit feedback, based on the curation task including deletion of an action, requires confirmation from the coder.

10. A system for calculating a risk adjustment factor (RAF), comprising one or more processors and one or more computer-readable storage devices having stored a plurality of computer-executable instructions, wherein execution of the computer-executable instructions causing the computer system to:
receive an input including a structured data and an unstructured data by a retrospective workflow to identify one or more clinical entities;
parse the unstructured data format into the structured data format by a Natural Language Processing (NLP) module and said parsed structured data and the input unstructured data is passed to an Application Recommendation System;
identify an evidence by the Application Recommendation System based upon the unstructured data and said evidence is processed based on an input received from a master knowledge graph module;
suggest an output by a prospective workflow to a coder, wherein the coder performs a plurality of curation tasks to provide a feedback to a feedback processing module; and
update the Application Recommendation System by adding a new rule therein and fine-tuning the risk adjustment workflow system,
wherein the retrospective workflow and the prospective workflow are provided as at least two stack block layers connected with each other and each stack block comprises a plurality of components, logic and flow.
